(19) United States
(12) Patent Application Publication
(10) Pub. No.: US 2003/0152972 A1
(43) Pub. Date: Aug. 14, 2003

(54) Gene expression associated with psychiatric disorders

(75) Inventors: Pamela Sklar, Brookline, MA (US); Tracey Petryshen, Cambridge, MA (US); Gloria Tsan, Brooklyn, NY (US); Joseph Lehar, Lexington, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Whitehead Institute for Biomedical Research, Cambridge, MA

(21) Appl. No.: 10/292,382

(22) Filed: Nov. 8, 2002

Related U.S. Application Data
(60) Provisional application No. 60/348,028, filed on Nov. 8, 2001.

(51) Int. Cl. C12Q 1/68

(52) U.S. Cl. 435/6

(57) ABSTRACT

The disclosure relates to methods of diagnosing psychiatric disorders (e.g., schizophrenia, bipolar disorder), methods of classifying a sample as derived from an individual having a psychiatric disorder, methods of identifying compounds for use in modulating psychiatric disorders, methods of modulating psychiatric disorders and methods of assessing efficacy of treatment of psychiatric disorders. The disclosure also relates to oligonucleotide microarrays containing probes for genes which are differentially expressed between schizophrenic individuals and normal individuals and to oligonucleotide microarrays containing probes for genes which are differentially expressed between bipolar individuals and normal individuals. The disclosure also relates to methods of classifying a sample as a pre-frontal cortex and/or cerebellum sample, as well as to oligonucleotide microarrays containing probes for genes which are differentially expressed in pre-frontal cortex and cerebellum.

### RELATED APPLICATION

This application claims the benefit of U.S. Provisional Application No. 60/348,028, filed Nov. 8, 2001. The entire teachings of the above application are incorporated herein by reference.

### GOVERNMENT SUPPORT

The invention was supported, in whole or in part, by grant number MH52168 from the National Institutes of Health. The Government has certain rights in the invention.

### BACKGROUND OF THE INVENTION

Classification of biological samples from individuals is not an exact science. In many instances, accurate diagnosis and safe and effective treatment of a disorder depend on being able to discern biological distinctions among cell or tissue samples from a particular area of the body. The classification of a sample from an individual into particular disease classes has often proven to be difficult, incorrect, or equivocal. Typically, using traditional methods such as histochemical analyses, immunophenotyping, and cytogenetic analyses, only one or two characteristics of the sample are analyzed to determine the sample's classification. Inaccurate results can lead to incorrect diagnoses and potentially ineffective or harmful treatment.

Neuropsychiatric disorders, such as schizophrenia, attention deficit disorders, schizoaffective disorders, bipolar disorders and unipolar disorders, differ from many other disorders in that anatomical or biochemical pathologies are often not readily detectable for neuropsychiatric disorders. Largely as a result of this difference, drugs which have been used to treat individuals with neuropsychiatric disorders, including lithium salts, valproic acid and carbamazepine, have not been predictably effective in treatment regimens across a variety of patients. Treatment regimens are further complicated by the fact that clinical diagnosis currently relies on clinical observation and subjective reports. Identification of the anatomical or biochemical defects which result in neuropsychiatric disorders is needed in order to effectively distinguish between the disorders and to allow the design and administration of effective therapeutics for these disorders.

### SUMMARY OF THE INVENTION

The invention relates to methods of diagnosing psychiatric disorders (e.g., schizophrenia, bipolar disorder), methods of classifying a sample as derived from an individual having a psychiatric disorder, methods of identifying compounds for use in modulating psychiatric disorders, methods of modulating psychiatric disorders and methods of assessing efficacy of treatment of psychiatric disorders. The invention also relates to oligonucleotide microarrays containing probes for genes which are differentially expressed between schizophrenic individuals and normal individuals and to oligonucleotide microarrays containing probes for genes which are differentially expressed between bipolar individuals and normal individuals. The present invention also relates to methods of classifying a sample as a pre-frontal cortex and/or cerebellum sample, as well as to oligonucleotide microarrays containing probes for genes which are differentially expressed in pre-frontal cortex and cerebellum.

In one embodiment, the invention relates to a method of diagnosing an individual as having a psychiatric disorder or at risk for developing a pyschiatric disorder comprising the steps of isolating a gene expression product from at least one informative gene from a biological sample from said individual; and determining the expression of at least one informative gene, wherein said expression is correlated with a psychiatric disorder, thereby diagnosing the individual. In a particular embodiment, expression of more than one informative gene can be determined to produce a gene expression profile of informative genes. In one embodiment, the psychiatric disorder is schizophrenia. In another embodiment, the psychiatric disorder is bipolar disorder. In one embodiment the sample is a prefrontal cortex sample.

In one embodiment of the invention, informative genes can be, for example, all or a subset of the genes shown in FIGS. 3, 4 and 5. In particular, the informative genes shown in FIG. 3 can be used to diagnose an individual as having schizophrenia or at risk for developing schizophrenia. For example, the expression of one or more genes shown in FIG. 3 can be determined in a sample from an individual to be assessed. If one or more of the genes shown in FIG. 3 is expressed at a reduced level in the individual as compared with a normal control, the individual is diagnosed as having schizophrenia or at risk for developing schizophrenia.

In another embodiment, the informative genes shown in FIGS. 4 and 5 can be used to diagnose an individual as having bipolar disorder or at risk for developing bipolar disorder. For example, the expression of one or more genes shown in FIGS. 4 and 5 can be determined in a sample from an individual to be assessed. If one or more of the genes shown in FIGS. 4 and 5 is expressed at an increased level in the individual as compared with a normal control, the individual is diagnosed as having bipolar disorder or at risk for developing bipolar disorder.

In one embodiment of the method, the gene expression product is mRNA, and in a particular embodiment, the gene expression profile is determined utilizing hybridization probes specific to one or more informative genes. In particular, the gene expression profile is determined utilizing oligonucleotide microarrays, containing probes or primers for all or a subset of the informative genes disclosed herein, immobilized on a solid support chip. In another embodiment of the invention, the gene expression product is a peptide, and in a particular embodiment, the gene expression profile is determined using antibodies.

In one embodiment, the invention relates to a method of classifying a sample as derived from an individual having a psychiatric disorder comprising the steps of isolating a gene expression product from at least one informative gene from one or more cells in said sample; and determining the expression of at least one informative gene, wherein said expression is correlated with a psychiatric disorder, thereby classifying the sample. In a particular embodiment, expression of more than one informative gene can be determined to produce a gene expression profile of informative genes. In one embodiment, the psychiatric disorder is schizophrenia. In another embodiment, the psychiatric disorder is bipolar disorder.

In one embodiment of the invention, informative genes can be, for example, all or a subset of the genes shown in FIGS. 3, 4 and 5. In particular, the informative genes shown in FIG. 3 can be used to classify a sample as derived from an individual having schizophrenia or at risk for developing schizophrenia. For example, the expression of one or more genes shown in FIG. 3 can be determined in the sample to be assessed. If one or more of the genes shown in FIG. 3 is expressed at a reduced level in the sample as compared with a normal control, the sample is classified as derived from an individual having schizophrenia or at risk for developing schizophrenia.

In another embodiment, the informative genes shown in FIGS. 4 and 5 can be used to classify a sample as derived from an individual having bipolar disorder or at risk for developing bipolar disorder. For example, the expression of one or more genes shown in FIGS. 4 and 5 can be determined in the sample to be assessed. If one or more of the genes shown in FIGS. 4 and 5 is expressed at an increased level in the sample as compared with a normal control, the sample is classified as derived from an individual having bipolar disorder or at risk for developing bipolar disorder.

In one embodiment of the method, the gene expression product is mRNA, and in a particular embodiment, the gene expression profile is determined utilizing hybridization probes specific to one or more informative genes. In particular, the gene expression profile is determined utilizing oligonucleotide microarrays, containing probes or primers for all or a subset of the informative genes disclosed herein, immobilized on a solid support chip. In another embodiment of the invention, the gene expression product is a peptide, and in a particular embodiment, the gene expression profile is determined using antibodies.

The invention also features a method of identifying a compound for use in modulating a psychiatric disorder, comprising the steps of: a) providing a cell or cell lysate sample; b) contacting the cell or cell lysate sample with a candidate compound; and c) detecting an increase in expression of at least one informative gene having decreased expression in an individual having a psychiatric disorder. A candidate compound that increases the expression of the informative gene is a compound for use in modulating the psychiatric disorder. In one embodiment, the cell or cell lysate sample is derived from pre-frontal cortex tissue. In another embodiment, the cell or cell lysate sample is derived from a cultured cell.

In other embodiments, gene expression is determined by assessing the DNA or mRNA level of the gene. Preferably, the DNA or mRNA level is determined utilizing specific hybridization probes. For example, the DNA or mRNA level may be determined utilizing oligonucleotide microarrays. In another embodiment, gene expression is determined by assessing the polypeptide level encoded by the informative gene. Preferably, gene expression is determined utilizing antibodies.

In a preferred embodiment, the one or more informative genes is selected from the group consisting of the genes in FIG. 3 and the psychiatric disorder is schizophrenia.

The invention also features a method of identifying a compound for use in modulating a psychiatric disorder, comprising the steps of: a) providing a cell or cell lysate sample; b) contacting the cell or cell lysate sample with a candidate compound; and c) detecting a decrease in expression of at least one informative gene having increased expression in an individual having a psychiatric disorder. A candidate compound that decreases the expression of the informative gene is a compound for use in modulating the psychiatric disorder. In one embodiment, the cell or cell lysate sample is derived from pre-frontal cortex tissue. In another embodiment, the cell or cell lysate sample is derived from a cultured cell.

In other embodiments, gene expression is determined by assessing the DNA or mRNA level of the gene. Preferably, the DNA or mRNA level is determined utilizing specific hybridization probes. For example, the DNA or mRNA level may be determined utilizing oligonucleotide microarrays. In another embodiment, gene expression is determined by assessing the polypeptide level encoded by the informative gene. Preferably, gene expression is determined utilizing antibodies.

In a preferred embodiment, the one or more informative genes is selected from the group consisting of the genes in FIGS. 4 and 5 and the psychiatric disorder is bipolar disorder.

The invention also features a method for modulating a psychiatric disorder in an individual comprising down-regulating (i.e., inhibiting) in the subject at least one informative gene shown to be expressed, or expressed in increased levels (as compared with a control), in individuals having a psychiatric disorder or at risk for developing a psychiatric disorder. In one embodiment, the psychiatric disorder is bipolar disorder and the at least one informative gene is selected from the group consisting of the genes in FIGS. 4 and 5.

The invention also features a method for modulating a psychiatric disorder in an individual comprising up-regulating (i.e., enhancing) in the subject at least one informative gene shown not to be expressed, or expressed at reduced levels (as comapred with a control), in individuals having a psychiatric disorder or at risk for developing a psychiatric disorder. In one embodiment, the psychiatric disorder is schizophrenia and the at least one informative gene is selected from the group consisting of the genes in FIG. 3.

The invention further relates to a method of assessing treatment efficacy in an individual having a psychiatric disorder comprising determining the expression level of one or more informative genes at multiple time points during treatment. In one embodiment, a decrease in expression of the one or more informative genes shown to be expressed, or expressed at increased levels (as compared with a control), in individuals having a psychiatric disorder or at risk for developing a psychiatric disorder, is indicative that treatment is effective. In one embodiment, the psychiatric disorder is bipolar disorder and the one or more informative genes are selected from the group consisting of the genes in FIGS. 4 and 5.

In another embodiment, an increase in expression of the one or more informative genes shown not to be expressed, or expressed at reduced levels (as compared with a control), in individuals having a psychiatric disorder or at risk for developing a psychiatric disorder, is indicative that treatment is effective. In one embodiment, the psychiatric disorder is schizophrenia and the one or more informative genes are selected from the group consisting of the genes in FIG. 3.

In one embodiment, the invention relates to a method of classifying a sample as derived from pre-frontal cortex or cerebellum comprising the steps of isolating a gene expression product from at least one informative gene from one or more cells in said sample; and determining the expression of at least one informative gene, wherein said expression is correlated with a sample derived from pre-frontal cortex or cerebellum, thereby classifying the sample. In a particular embodiment, expression of more than one informative gene can be determined to produce a gene expression profile of informative genes. In one embodiment of the invention, informative genes can be, for example, all or a subset of the genes shown in FIGS. 1 and 2.

In one embodiment of the method, the gene expression product is mRNA, and in a particular embodiment, the gene expression profile is determined utilizing hybridization probes specific to one or more informative genes. In particular, the gene expression profile is determined utilizing oligonucleotide microarrays, containing probes or primers for all or a subset of the informative genes disclosed herein, immobilized on a solid support chip. In another embodiment of the invention, the gene expression product is a peptide, and in a particular embodiment, the gene expression profile is determined using antibodies.

The invention also features oligonucleotide microarrays having immobilized thereon a plurality of oligonucleotide probes specific for one or more informative genes selected from the group consisting of the genes in FIGS. 1A-1D, 2A-2E, 3, 4 and 5.

### BRIEF DESCRIPTION OF THE DRAWINGS

FIGS. 1A-1D show the top 100 genes that have increased expression in frontal cortex compared to cerebellum (>99% significance level) according to the work described herein.

FIGS. 2A-2E show the top 100 genes that have increased expression in cerebellum compared to frontal cortex (>99% significance level) according to the work described herein.

FIG. 3 shows genes that have increased expression in control frontal cortex compared to schizophrenia frontal cortex (>95% significance level) according to the work described herein.

FIG. 4 shows the genes that have increased expression in bipolar frontal cortex compared to control frontal cortex (>99% significance level) according to the work described herein.

FIG. 5 shows the genes that have increased expression in bipolar frontal cortex compared to control frontal cortex (>95% significance level) according to the work described herein.

### DETAILED DESCRIPTION OF THE INVENTION

To identify genes involved in psychiatric disorders, microarray expression profiling was performed using microarrays able to query approximately 12,000 genes on post-mortem prefrontal cortex (area 46) and cerebellum samples from 15 each of control, schizophrenia, bipolar disorder, and depression patients. Initially all samples from prefrontal cortex and cerebellum were investigated, and it was possible to distinguish these morphologically and functionally different regions by their distinct gene expression profiles. In particular, high prefrontal cortex expression of neurogranin and human brain factor-1, and high cerebellum expression of Zic and NeuroD, were detected, consistent with the known regional expression patterns of these genes. These results indicate that the methods are robust. A minority of samples did not demonstrate region-specific expression profiles, suggestive of sample or experimental error, and these samples were discarded from further analyses.

Investigation of schizophrenia patient expression profiles identified twelve genes with significantly decreased expression in prefrontal cortex compared to controls (95% significance level under random permutation testing). The most significantly decreased gene, Fe65/APBB1, is known to interact with the beta-amyloid precursor protein, and is implicated in transcription and cell motility. No genes had significantly increased expression in schizophrenia prefrontal cortex compared to controls, nor were there any genes differentially expressed between schizophrenia and control cerebellum.

Investigation of bipolar disorder patient expression profiles identified two genes with significantly increased expression (99% significance level) in bipolar prefrontal cortex: human kinesin-like motor protein, involved in vesicle transport, and ryudocan (syndecan-4), an endothelial heparin sulfate proteoglycan. In addition, 15 genes had increased bipolar prefrontal cortex expression at the 95% significance level. No genes had significantly decreased expression in bipolar prefrontal cortex compared to controls, and no genes were differentially expressed between bipolar disorder and control cerebellum.

In one embodiment, the invention relates to a method of diagnosing an individual as having a psychiatric disorder or at risk for developing a pyschiatric disorder comprising the steps of isolating a gene expression product from at least one informative gene from a biological sample from said individual; and determining the expression of at least one informative gene, wherein said expression is correlated with a psychiatric disorder, thereby diagnosing the individual. In a particular embodiment, expression of more than one informative gene can be determined to produce a gene expression profile of informative genes. In one embodiment, the psychiatric disorder is schizophrenia. In another embodiment, the psychiatric disorder is bipolar disorder. In one embodiment the sample is a prefrontal cortex sample.

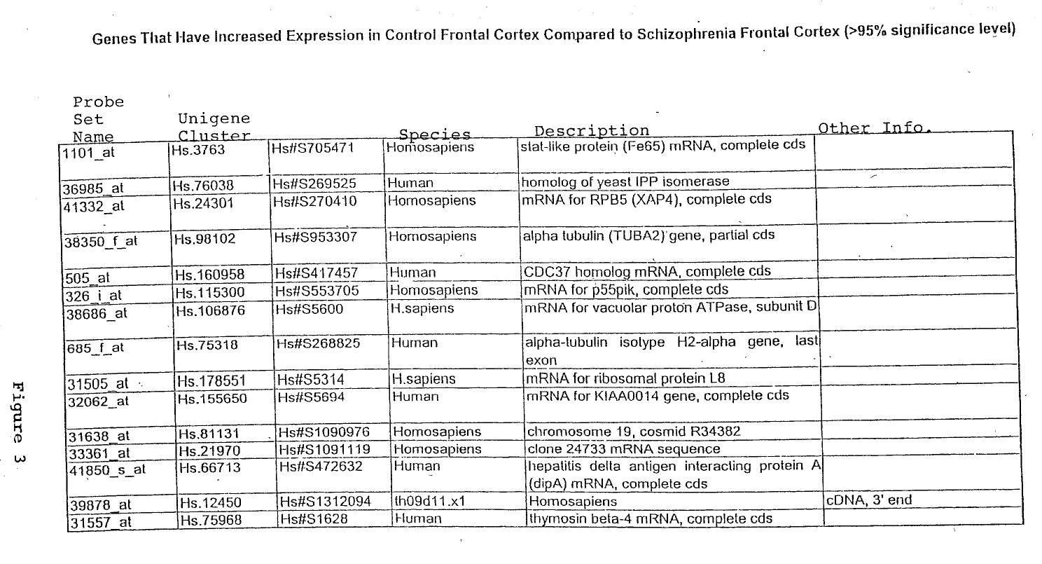

In one embodiment of the invention, informative genes can be, for example, all or a subset of the genes shown in FIGS. 3, 4 and 5. In particular, the informative genes shown in FIG. 3 can be used to diagnose an individual as having schizophrenia or at risk for developing schizophrenia. For example, the expression of one or more genes shown in FIG. 3 can be determined in a sample from an individual to be assessed. If one or more of the genes shown in FIG. 3 is expressed at a reduced level in the individual as compared with a normal control, the individual is diagnosed as having schizophrenia or at risk for developing schizophrenia.

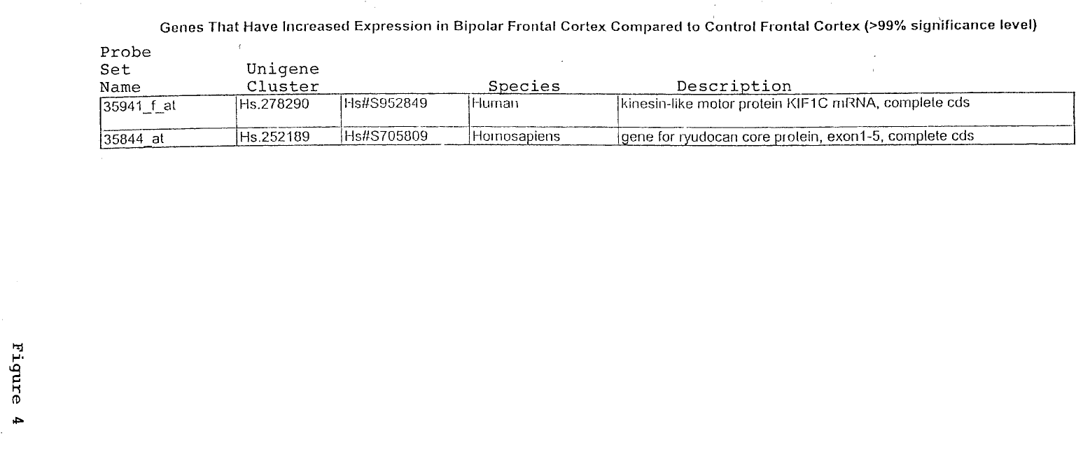

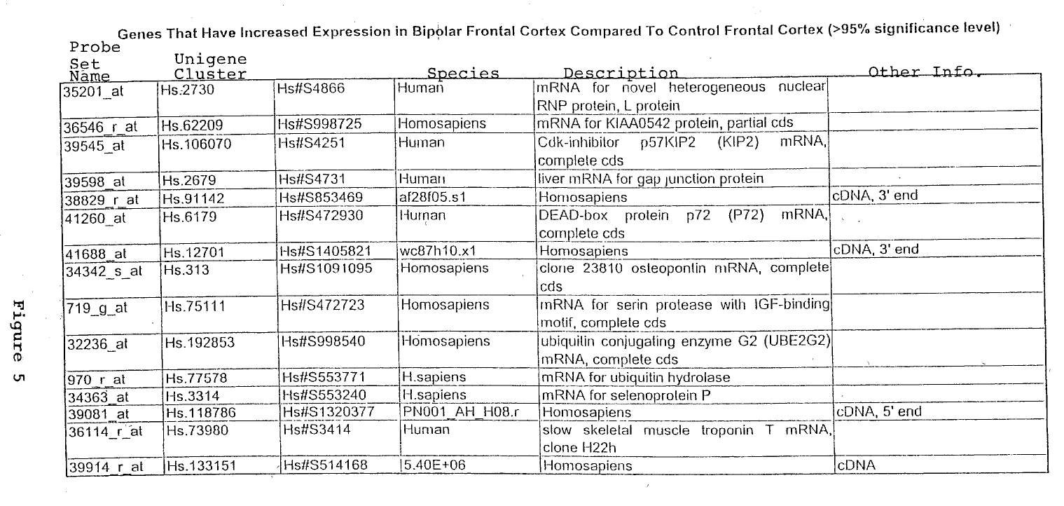

In another embodiment, the informative genes shown in FIGS. 4 and 5 can be used to diagnose an individual as having bipolar disorder or at risk for developing bipolar disorder. For example, the expression of one or more genes shown in FIGS. 4 and 5 can be determined in a sample from an individual to be assessed. If one or more of the genes shown in FIGS. 4 and 5 is expressed at an increased level in the individual as compared with a normal control, the individual is diagnosed as having bipolar disorder or at risk for developing bipolar disorder.

In one embodiment of the method, the gene expression product is mRNA, and in a particular embodiment, the gene expression profile is determined utilizing hybridization probes specific to one or more informative genes. In particular, the gene expression profile is determined utilizing oligonucleotide microarrays, containing probes or primers for all or a subset of the informative genes disclosed herein, immobilized on a solid support chip. In another embodiment of the invention, the gene expression product is a peptide, and in a particular embodiment, the gene expression profile is determined using antibodies.

By “gene expression profile” is meant the level or amount of gene expression of particular genes, for example, informative genes, as assessed by methods described herein. The gene expression profile can comprise data for one or more informative genes and can be measured at a single time point or over a period of time. For example, the gene expression profile can be determined using a single informative gene, or it can be determined using two or more informative genes, three or more informative genes, five or more informative genes, ten or more informative genes, twenty-five or more informative genes, or fifty or more informative genes. A gene expression profile may include expression levels of genes that are not informative, as well as informative genes. Phenotype classification (e.g., the presence or absence of a psychiatric disorder) can be made by comparing the gene expression profile of the sample with respect to one or more informative genes with one or more gene expression profiles (e.g., in a database). Using the methods described herein, expression of numerous genes can be measured simultaneously. The assessment of numerous genes provides for a more accurate evaluation of the sample because there are more genes that can assist in classifying the sample. A gene expression profile may involve only those genes that are increased in expression in a sample, only those genes that are decreased in expression in a sample, or a combination of genes that are increased and decreased in expression in a sample.

As used herein, “informative genes,” refers to a gene or genes whose expression correlates with a particular phenotype. Expression profiles obtained for informative genes can be used to determine, for example, the presence or absence of a psychiatric disorder in an individual, or whether a candidate compound increases or decreases gene expression in a sample. Samples can be classified according to their broad expression profile, or according to the expression levels of particular informative genes. The genes that are relevant for classification are referred to herein as “informative genes.” Not all informative genes for a particular class or phenotype distinction must be assessed in order to classify a sample. Similarly, the set of informative genes that characterize one phenotypic effect may or may not be the same as the set of informative genes for a different phenotypic effect. For example, a subset of the informative genes that demonstrate a high correlation with a distinction between pre-frontal cortex and cerebellum can be used in classifying a sample with regard to brain origin. This subset can be, for example, one or more genes, two or more genes, three or more genes, five or more genes, ten or more genes, twenty-five or more genes, or fifty or more genes. The informative genes that characterize other classification categories can be the same or different from the informative genes that characterize cortex or cerebellum. Typically the accuracy of the classification increases with the number of informative genes that are assessed.

As used herein, “gene expression products” are proteins, polypeptides, or nucleic acid molecules (e.g., mRNA, tRNA, rRNA, cDNA, or cRNA) that result from transcription or translation of genes. The present invention can be used effectively to analyze proteins, polypeptides, or nucleic acid molecules that are the result of transcription or translation, particularly of informative genes identified herein. The nucleic acid molecule levels measured can be derived directly from the gene or, alternatively, from a corresponding regulatory gene or regulatory sequence element. All forms of gene expression products can be measured. For example, the nucleic acid molecule can be transcribed to obtain an RNA gene expression product. If desired, the transcript can be translated using, for example, standard in vitro translation methods to obtain a polypeptide gene expression product. Polypeptide gene expression products can be used in protein binding assays, for example, antibody assays, or in nucleic acid binding assays, standardly known in the art, in order to identify a psychiatric disorder or compounds involved in the development or modulation of a psychiatric disorder. Additionally, variants of genes and gene expression products including, for example, spliced variants and polymorphic alleles, can be measured. Similarly, gene expression can be measured by assessing the level of a polypeptide or protein or derivative thereof translated from mRNA. The sample to be assessed can be any sample that contains a gene expression product. Suitable sources of gene expression products, e.g., samples, can include intact cells, lysed cells, cellular material for determining gene expression, or material containing gene expression products. Examples of such samples are brain tissue, cells derived from brain tissue (e.g., pre-frontal cortex tissue and/or the cerebellum), nucleic acids or polypeptides derived from brain tissue, or other cell samples. Methods of obtaining such samples are known in the art.

By “increased expression” is meant the level of a gene expression product is made higher and/or the activity of the gene expression product is enhanced. Preferably, the increase is by at least 1.5-fold, more preferably the increase is at least 2-fold, 5-fold, or 10-fold, and most preferably, the increase is at least 20-fold, relative to a control.

By “decreased expression” is meant the level of a gene expression product is made lower and/or the activity of the gene expression product is lowered. Preferably, the decrease is at least 25%, more preferably, the decrease is at least 50%, 60%, 70%, 80%, or 90% and most preferably, the decrease is at least one-fold, relative to a control.

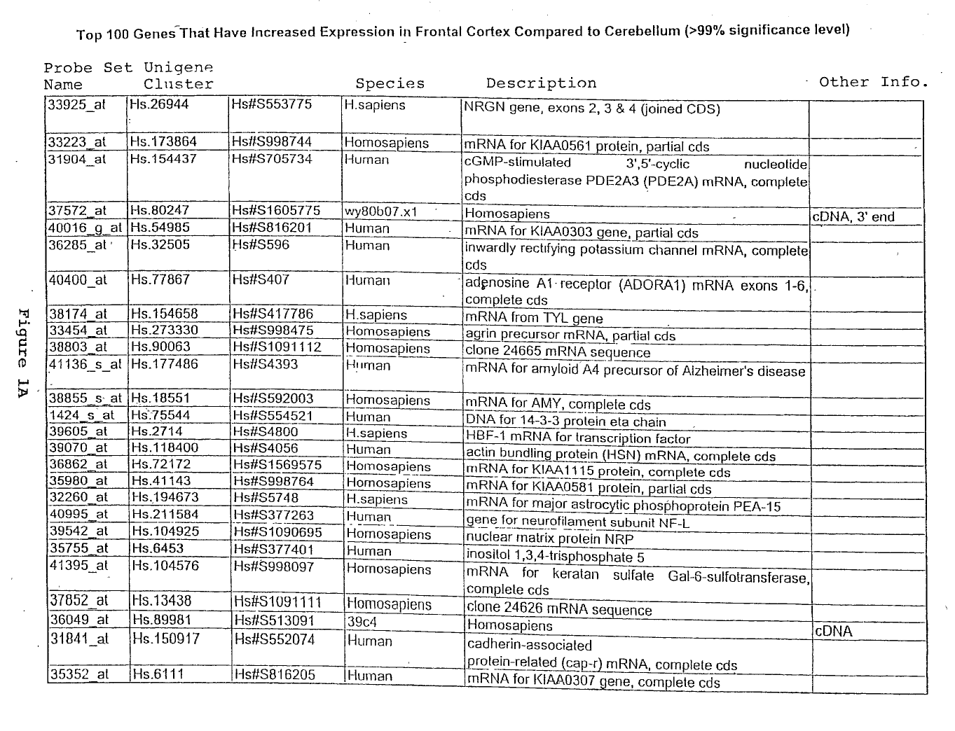

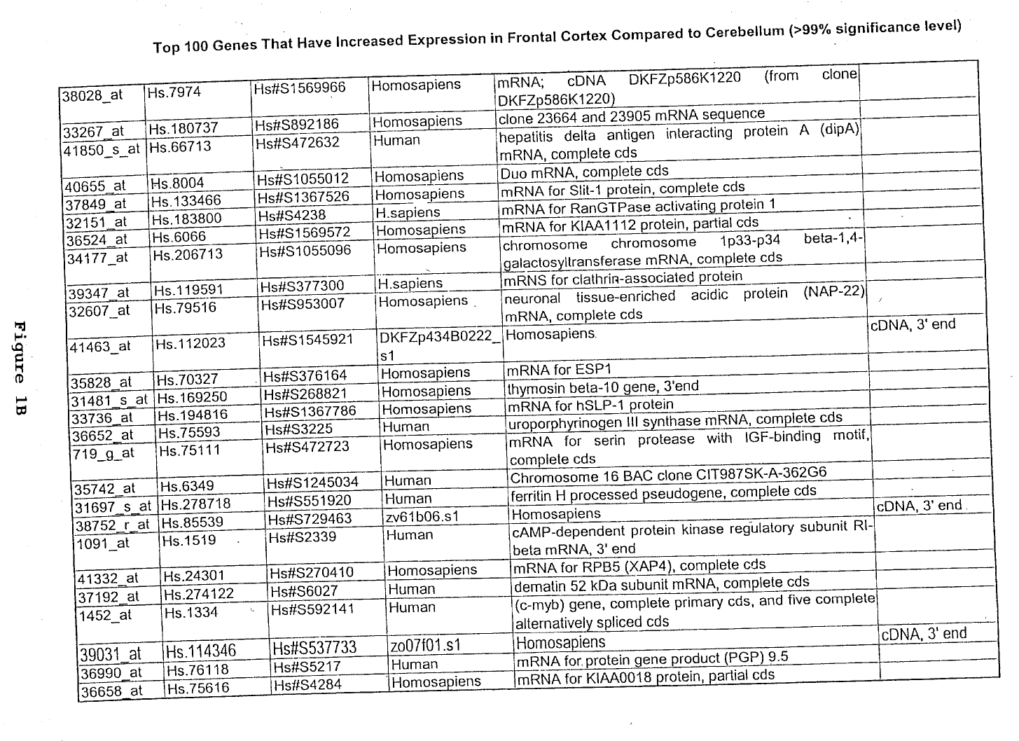

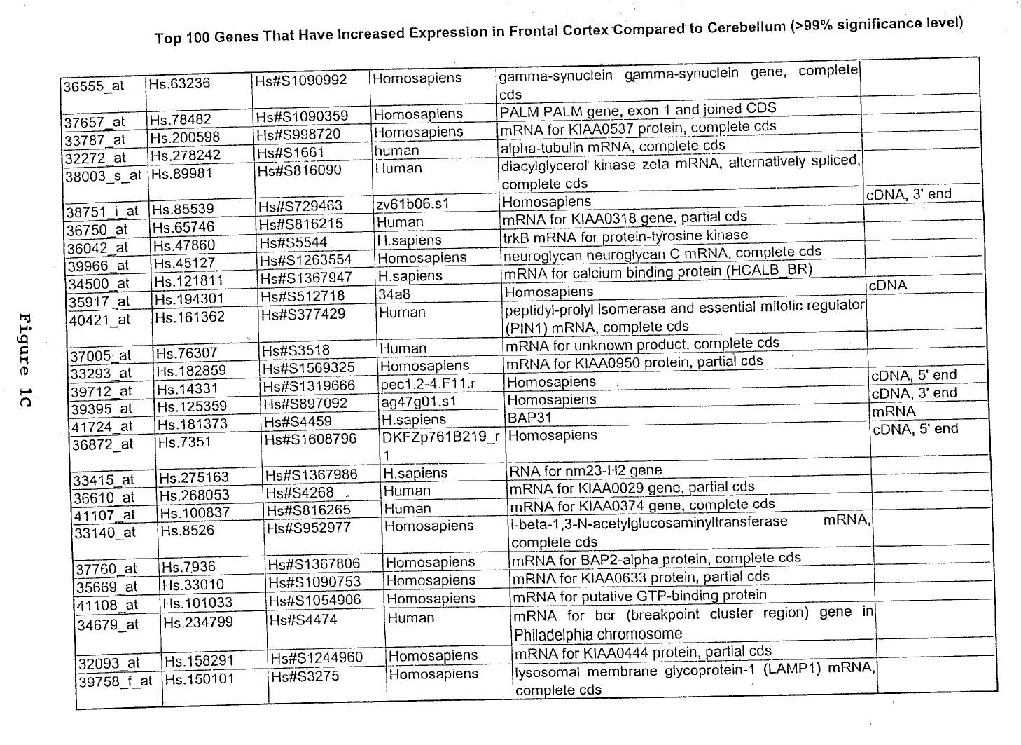

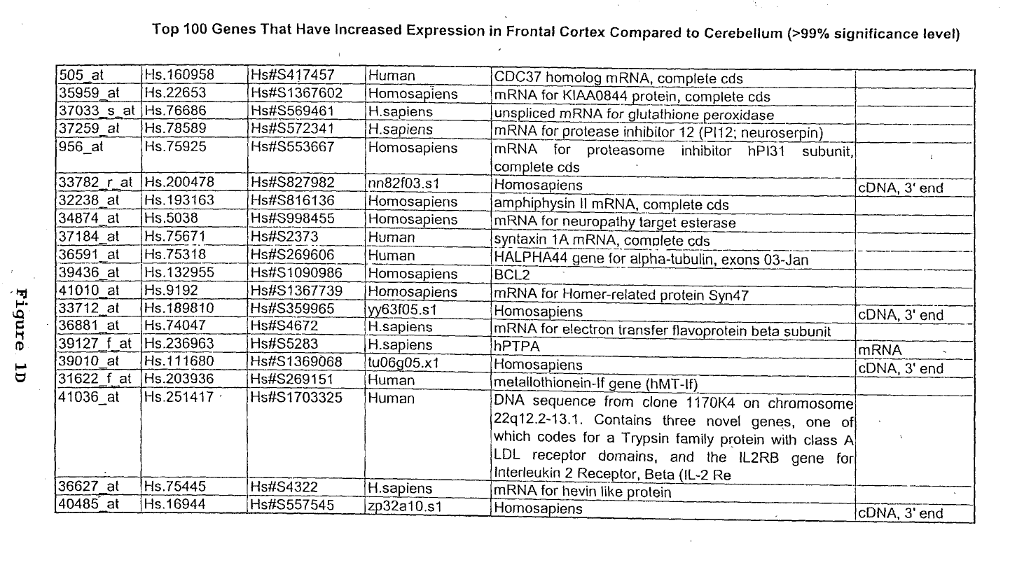

Genes that are particularly relevant for classification, i.e., demonstrate a different expression profile in different classification categories, have been identified as a result of work described herein and are shown in FIGS. 1A-1D, 2A-2E, 3, 4 and 5.

In one embodiment, the gene expression product is a protein or polypeptide. As used herein, by “polypeptide” is meant any chain of more than two amino acids, regardless of post-translational modification such as glycosylation or phosphorylation. Examples of polypeptides include, but are not limited to, proteins. In this embodiment the determination of the gene expression profile is made using techniques for protein detection and quantitation known in the art. For example, antibodies that specifically interact with the protein or polypeptide expression product of one or more informative genes can be obtained using methods that are routine in the art. The specific binding of such antibodies to protein or polypeptide gene expression products can be detected and measured by methods known in the art, for example, Western blot analysis or ELISA techniques.

In a preferred embodiment, the gene expression product is a nucleic acid, for example, DNA or mRNA, and the gene expression levels are obtained by contacting the sample with a suitable microarray on which probes specific for all or a subset of the informative genes have been immobilized, and determining the extent of hybridization of the nucleic acid in the sample to the probes on the microarray. Such microarrays are also within the scope of the invention. Examples of methods of making oligonucleotide microarrays are described, for example, in WO 95/11995. Other methods are readily known to the skilled artisan.

The gene expression value measured or assessed is the numeric value obtained from an apparatus that can measure gene expression levels. Gene expression levels refer to the amount of expression of the gene expression product, as described herein. The values are raw values from the apparatus, or values that are optionally re-scaled, filtered and/or normalized. Such data is obtained, for example, from a GeneChip® probe array or Microarray (Affymetrix, Inc.; U.S. Pat. Nos. 5,631,734, 5,874,219, 5,861,242, 5,858,659, 5,856,174, 5,843,655, 5,837,832, 5,834,758, 5,770,722, 5,770,456, 5,733,729, 5,556,752, all of which are incorporated herein by reference in their entirety), and the expression levels are calculated with software (e.g., Affymetrix GENECHIP software). For example, nucleic acids (e.g., mRNA or DNA) from a sample that has been subjected to particular stringency conditions hybridize to the probes on the chip. The nucleic acid to be analyzed (e.g., the target) is isolated, amplified and labeled with a detectable label, (e.g., ³²P or fluorescent label) prior to hybridization to the arrays. After hybridization, the arrays are inserted into a scanner that can detect patterns of hybridization. These patterns are detected by detecting the labeled target now attached to the microarray, e.g., if the target is fluorescently labeled, the hybridization data are collected as light emitted from the labeled groups. Since labeled targets hybridize, under appropriate stringency conditions known to one of skill in the art, specifically to complementary oligonucleotides contained in the microarray, and since the sequence and position of each oligonucleotide in the array are known, the identity of the target nucleic acid applied to the probe is determined.

Quantitation of gene profiles from the hybridization of a labeled nucleic acid microarray can be performed by scanning the microarray to measure the amount of hybridization at each position on the microarray with an Affymetrix scanner (Affymetrix, Santa Clara, Calif.). For each stimulus a time series of nucleic acid levels (C={C1,C2,C3, . . . Cn}) and a corresponding time series of nucleic acid levels (M={M1,M2,M3, . . . Mn}) in control medium in the same experiment as the stimulus is obtained. Quantitative data is then analyzed. Hybridization analysis using microarray is only one method for obtaining gene expression values. Other methods for obtaining gene expression values known in the art or developed in the future can be used with the present invention. Once the gene expression values are determined, the sample can be classified.

Once the gene expression levels of the sample are obtained, the levels are compared or evaluated against a model or control sample(s), and then the sample is classified, for example, based one whether a particular informative gene in the sample exhibits increased or decreased expression. The evaluation of the sample determines whether or not the sample is assigned to a particular phenotypic class, for example, whether or not the sample is derived from an individual having a psychiatric disorder or whether or not a candidate compound modulates a psychiatric disorder.

The correlation between gene expression and class distinction can be determined using a variety of methods. Methods for defining classes and classifying samples are described, for example, in U.S. patent application Ser. No. 09/544,627, filed Apr. 6, 2000 by Golub et al., the teachings of which are incorporated herein by reference in their entirety. The information provided by the present invention, alone or in conjunction with other test results, aids in sample classification.

The present invention also features methods for identifying compounds that modulate psychiatric disorders. Novel compounds identified as described herein are also the subject of the invention. The invention also features a method of identifying a compound for use in modulating a psychiatric disorder, comprising the steps of: a) providing a cell or cell lysate sample; b) contacting the cell or cell lysate sample with a candidate compound; and c) detecting an increase in expression of at least one informative gene having decreased expression in an individual having a psychiatric disorder. A candidate compound that increases the expression of the informative gene is a compound for use in modulating the psychiatric disorder. In one embodiment, the cell or cell lysate sample is derived from pre-frontal cortex tissue. In another embodiment, the cell or cell lysate sample is derived from a cultured cell.

An increase or decrease in an informative gene may be identified using any of the methods described herein (or any analogous method known in the art). For example, oligonucleotide array systems described herein may be used to determine whether the addition of a test compound to a sample increases or decreases expression of an informative gene in that sample.

In other embodiments, gene expression is determined by assessing the DNA or mRNA level of the gene. Preferably, the DNA or mRNA level is determined utilizing specific hybridization probes. For example, the DNA or mRNA level may be determined utilizing oligonucleotide microarrays. In another embodiment, gene expression is determined by assessing the polypeptide level encoded by the informative gene. In this embodiment, gene expression can be determined, for example, utilizing antibodies.

In a preferred embodiment, the one or more informative genes is selected from the group consisting of the genes in FIG. 3 and the psychiatric disorder is schizophrenia.

The invention also features a method of identifying a compound for use in modulating a psychiatric disorder, comprising the steps of: a) providing a cell or cell lysate sample; b) contacting the cell or cell lysate sample with a candidate compound; and c) detecting a decrease in expression of at least one informative gene having increased expression in an individual having a psychiatric disorder. A candidate compound that decreases the expression of the informative gene is a compound for use in modulating the psychiatric disorder. In one embodiment, the cell or cell lysate sample is derived from pre-frontal cortex tissue. In another embodiment, the cell or cell lysate sample is derived from a cultured cell.

In other embodiments, gene expression is determined by assessing the DNA or mRNA level of the gene. Preferably, the DNA or mRNA level is determined utilizing specific hybridization probes. For example, the DNA or mRNA level may be determined utilizing oligonucleotide microarrays. In another embodiment, gene expression is determined by assessing the polypeptide level encoded by the informative gene. In this embodiment, gene expression can be determined, for example, utilizing antibodies.

In a preferred embodiment, the one or more informative genes is selected from the group consisting of the genes in FIGS. 4 and 5 and the psychiatric disorder is bipolar disorder.

By “modulating a psychiatric disorder” is meant increasing or decreasing the occurrence or severity of the disorder in an individual. The modulation may be the result of contacting a sample (for example, a cell, tissue, cell or tissue lysate, nucleic acid, or polypeptide) with a candidate compound. It will be appreciated that the degree of modulation provided by a candidate compound in a given assay will vary, but that one skilled in the art can determine the statistically significant change or a therapeutically effective change in the occurrence or severity of the disorder.

By a “candidate compound” is meant a molecule, be it naturally-occurring or artificially derived, that is surveyed or assessed for its effects on the gene expression profile of an informative gene, employing methods described herein. Examples of candidate compounds include, but are not limited to peptides, polypeptides, synthetic organic molecules, naturally occurring organic molecules, nucleic acid molecules, and combinations thereof.

By “increasing gene expression” is meant raising the level of expression, and/or the activity, of one or more informative genes in a cell, tissue, cell lysate, or tissue lysate sample relative to a control sample. An increase in gene expression may occur, for example, when the sample is contacted with a candidate compound. The control sample may be a cell, tissue, individual, cell lysate, or tissue lysate that was not contacted or treated with the candidate compound or that was contacted with candidate compound vehicle only. Preferably, the increase is at least 1.5-fold, more preferably the increase is at least 2-fold, 5-fold, or 10-fold, and most preferably, the increase is at least 20-fold, relative to a control sample.

By “decreasing gene expression” is meant lowering the level or expression of, and/or the activity of, one or more informative genes in a cell, tissue, cell lysate, or tissue lysate sample relative to a control sample. A decrease in gene expression may occur, for example, when the sample is contacted with a candidate compound. The control sample may be a cell, tissue, cell lysate, or tissue lysate that was not contacted with the candidate compound or that was contacted with candidate compound vehicle only. Preferably, the decrease in gene expression of an informative gene is at least 25%, more preferably, the decrease is at least 50%, 60%, 70%, 80%, or 90% and most preferably, the decrease is at least one-fold, relative to a control sample.

The expression level of an informative gene may be modulated by modulating transcription, translation, or mRNA or protein turnover, or the activity of the gene expression product, and such modulation may be detected using known methods for measuring mRNA and protein levels and activities, e.g., oligonucleotide microarray hybridization, RT-PCR, and ELISA and nucleic acid and protein binding assays.

A compound that increases the expression level of a gene that is decreased in a psychiatric disorder can be useful for treating psychiatric disorders. In addition, a compound that decreases the expression level of a gene that is increased in a psychiatric disorder can also be useful for treating psychiatric disorders.

While the above described candidate compound screening methods are designed primarily to identify candidate compounds that may be used to decrease the occurrence or severity of psychiatric disorders, identification of candidate compounds that increase the occurrence or severity of psychiatric disorders is also a feature of the present invention. Such candidate compound identification methods involve contacting a sample, for example, a cell, cell lysate, tissue, or tissue lysate with a candidate compound, and detecting an increase in expression of at least one informative gene having increased expression in a psychiatric disorder. A candidate compound that increases expression of such an informative gene is a compound for use in modulating a psychiatric disorder.

Alternatively, a compound that modulates a psychiatric disorder can be identified by contacting a sample, for example, a cell, cell lysate, tissue, individual, or tissue lysate with a candidate compound, and detecting a decrease in expression of at least one informative gene having decreased expression in a psychiatric disorder. A candidate compound that decreases expression of such an informative gene is a compound for use in modulating the psychiatric disorder. These candidate compound identification methods may be used for identifying compounds that increase the severity or occurrence of psychiatric disorders. Such compounds may be identified as compounds to which exposure should be minimized in order to decrease one's likelihood of developing a psychiatric disorder.

In general, novel drugs for modulation of psychiatric disorders can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available, e.g., Chembridge (San Diego, Calif.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are generated, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their psychiatric disorder-modulatory activities should be employed whenever possible.

When a crude extract is found to modulate (i.e., stimulate (increase) or inhibit (decrease)) a psychiatric disorder/development of a psychiatric disorder, further fractionation of the positive lead extract is desirable to isolate chemical constituents responsible for the observed effect. Thus, the goal of the extraction, fractionation, and purification process is the careful characterization and identification of a chemical entity within the crude extract having an activity that increases or deceases. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases.

The invention further relates to a method of assessing treatment efficacy in an individual having a psychiatric disorder comprising determining the expression level of one or more informative genes at multiple time points during treatment. In one embodiment, a decrease in expression of the one or more informative genes shown to be expressed, or expressed at increased levels (as compared with a control), in individuals having a psychiatric disorder or at risk for developing a psychiatric disorder, is indicative that treatment is effective. In one embodiment, the psychiatric disorder is bipolar disorder and the one or more informative genes are selected from the group consisting of the genes in FIGS. 4 and 5.

In another embodiment, an increase in expression of the one or more informative genes shown not to be expressed, or expressed at reduced levels (as compared with a control), in individuals having a psychiatric disorder or at risk for developing a psychiatric disorder, is indicative that treatment is effective. In one embodiment, the psychiatric disorder is schizophrenia and the one or more informative genes are selected from the group consisting of the genes in FIG. 3.

Informative genes identified as described herein can also be targeted in methods of modulating psychiatric disorders. For example, expression of at least one informative gene shown to be expressed in an individual having a psychiatric disorder or expressed in increased levels (as compared with a control), can be down-regulated in a method of inhibiting a psychiatric disorder. Alternatively, expression of at least one informative gene shown not to be expressed in an individual having a psychiatric disorder or which is expressed in reduced levels (as compared with a control) can be upregulated in a method of inhibiting a psychiatric disorder. Compounds identified by methods described herein, for example, can be utilized in methods of treatment of psychiatric disorders.

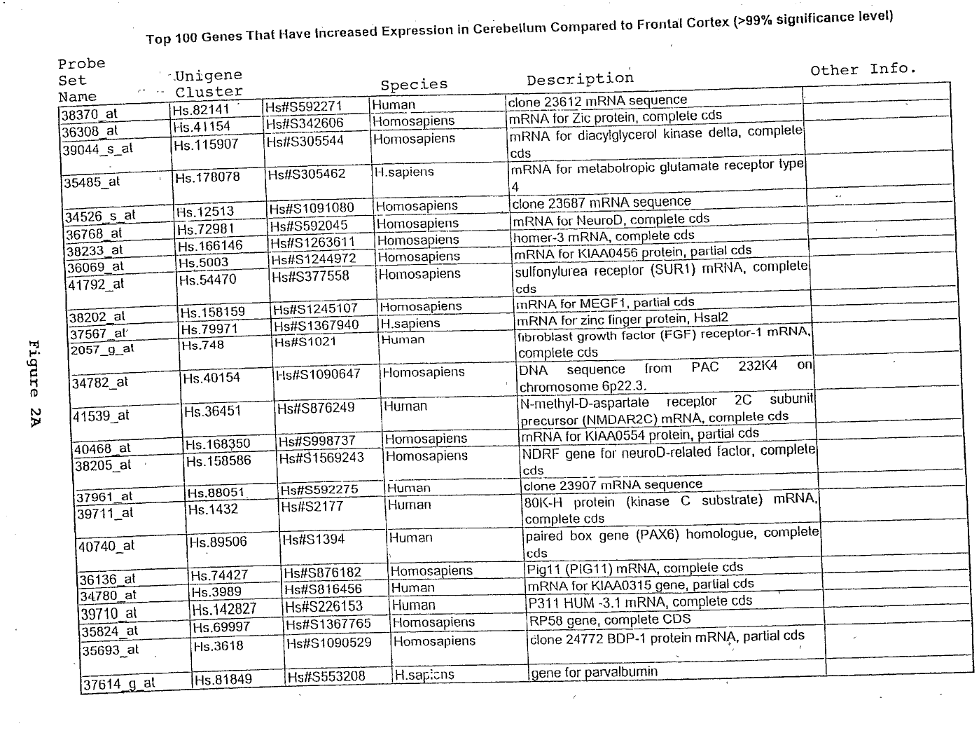

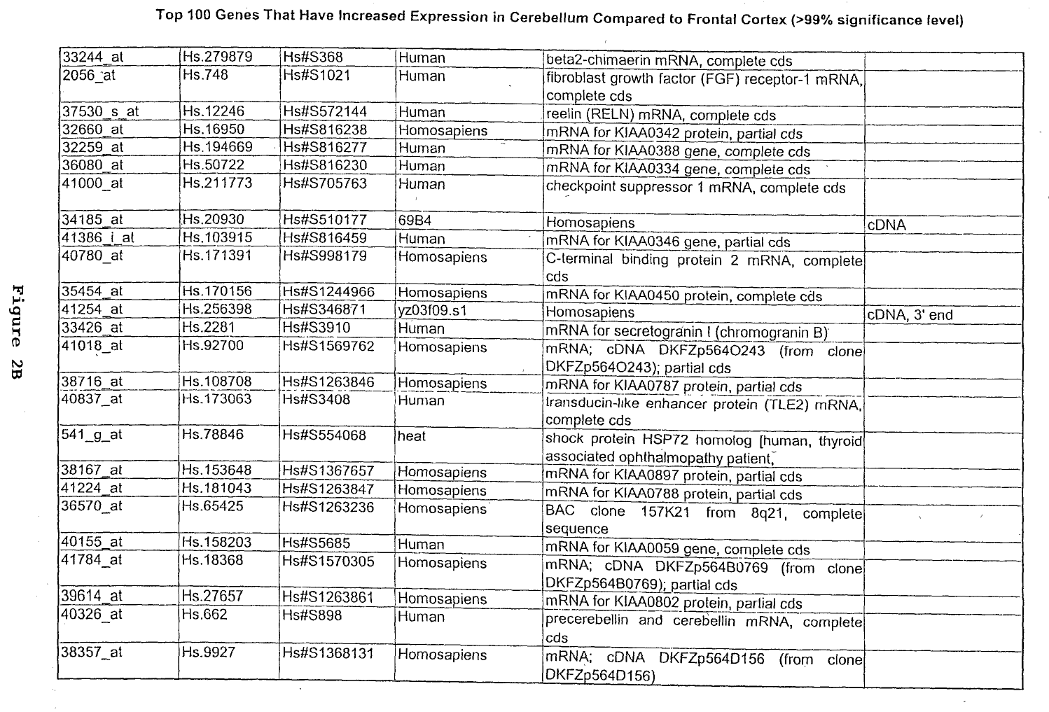

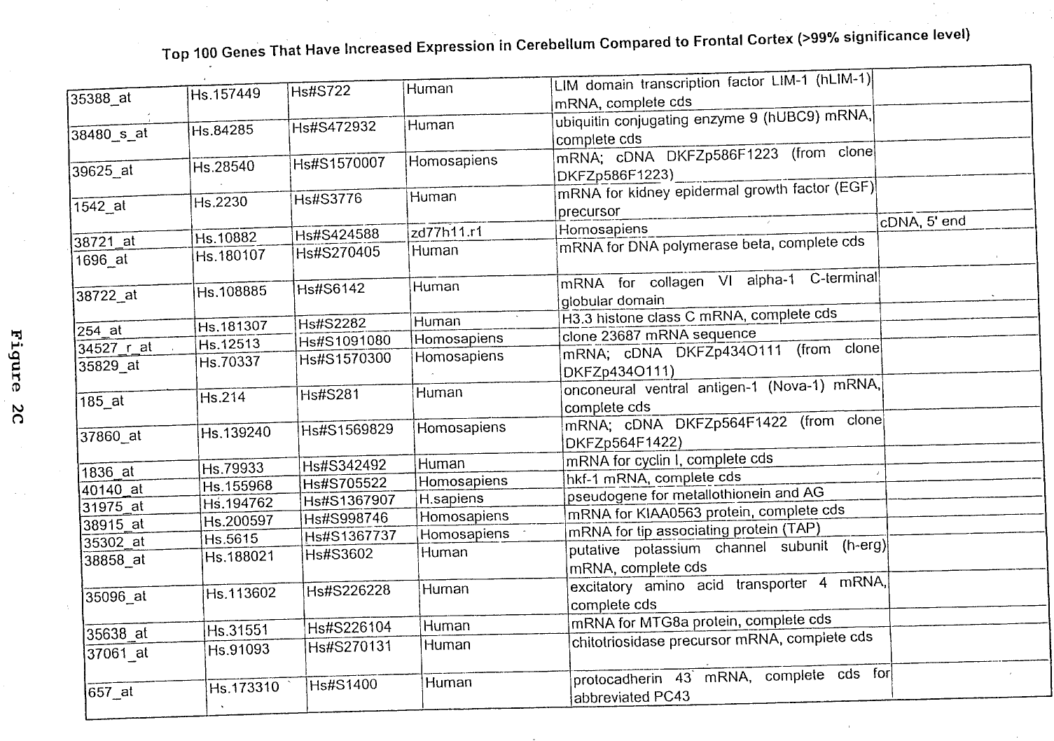

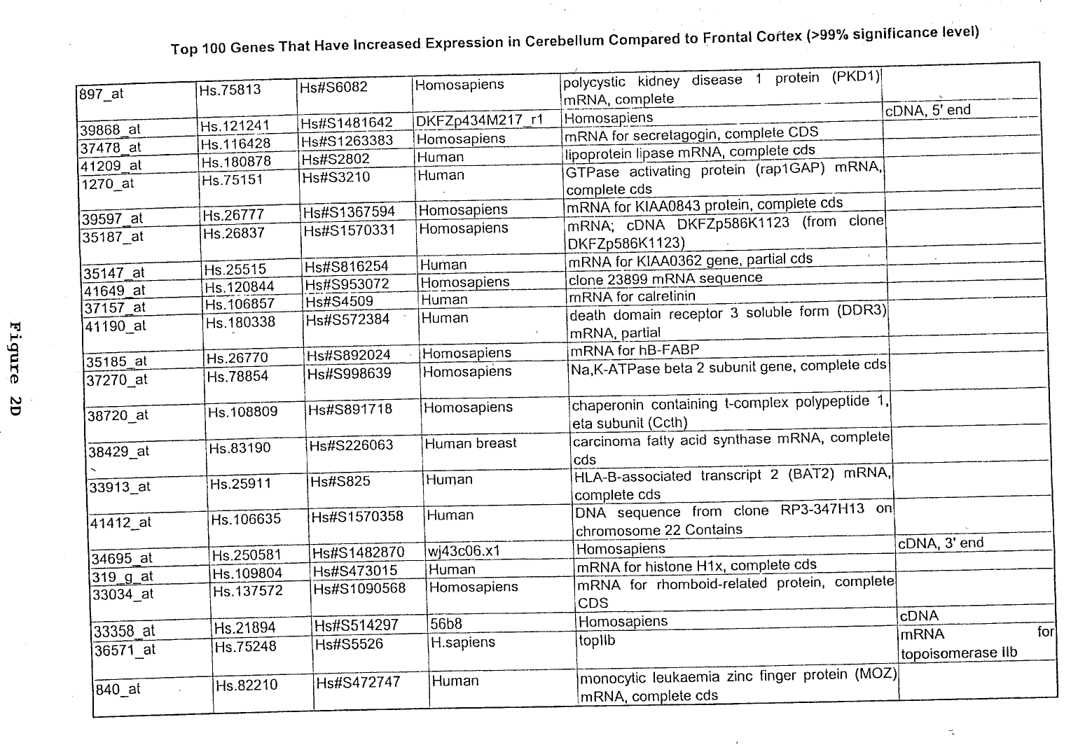

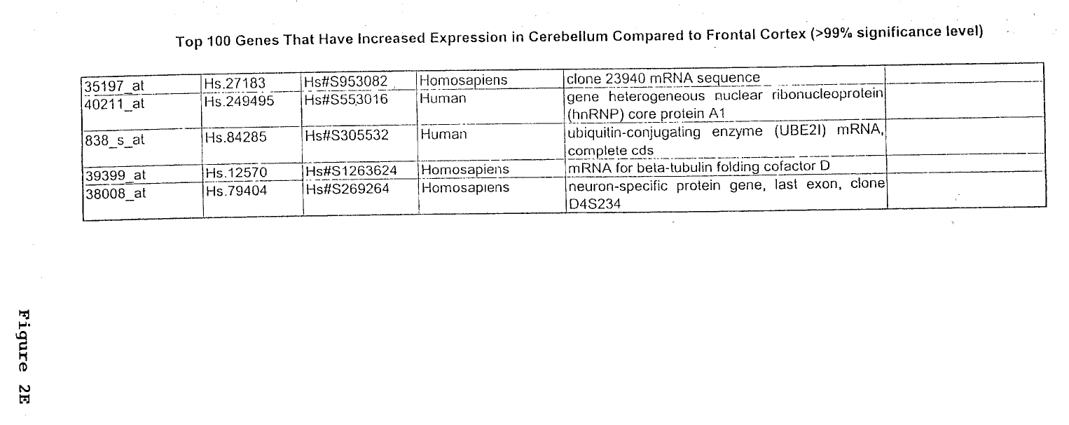

The present invention also features arrays, for example, microarrays that have a plurality of oligonucleotide probes for informative genes identified herein immobilized thereon. The oligonucleotide probe may be specific for one or more informative genes, selected from those in FIGS. 1A-1D, 2A-2E 3, 4 and 5. Methods for making oligonucleotide microarrays are well known in the art, and are described, for example, in WO 95/11995, the entire teachings of which are hereby incorporated by reference.

The present invention also provides information regarding the genes that are important in psychiatric disorders, thereby providing additional targets for diagnosis and therapy. It is clear that the present invention can be used to generate databases comprising informative genes that will have many applications in medicine, research and industry; such databases are also within the scope of the invention.

The teachings of all the patents, patent applications and all other publications and web sites cited herein are incorporated by reference in their entirety.

What is claimed is:

1. A method of diagnosing an individual as having a psychiatric disorder or at risk for developing a pyschiatric disorder comprising the steps of:
a) isolating a gene expression product from at least three informative genes from a biological sample from said individual; and
b) determining the expression of at least three informative genes,
wherein said expression is correlated with a psychiatric disorder or at risk for developing a pyschiatric disorder, thereby diagnosing the individual.

2. A method of diagnosing an individual as having schizophrenia or at risk for developing schizophrenia comprising the steps of:
a) isolating a gene expression product from at least three informative genes shown in FIG. 3 from a biological sample from said individual; and
b) determining the expression of at least three informative genes,
wherein if the informative genes are expressed at a reduced level in the individual as compared with a normal control, the individual is diagnosed as having schizophrenia or at risk for developing schizophrenia.

3. A method of diagnosing an individual as having bipolar disorder or at risk for developing bipolar disorder comprising the steps of:
a) isolating a gene expression product from at least three informative genes shown in FIGS. 4 and 5 from a biological sample from said individual; and
b) determining the expression of at least three informative genes,
wherein if the informative genes are expressed at an increased level in the individual as compared with a normal control, the individual is diagnosed as having bipolar disorder or at risk for developing bipolar disorder.

4. A method of classifying a sample as derived from an individual having a psychiatric disorder or at risk for developing a psychiatric disorder comprising the steps of:
a) isolating a gene expression product from at least three informative genes from one or more cells in said sample; and
b) determining the expression of at least three informative genes,
wherein said expression is correlated with a psychiatric disorder, thereby classifying the sample.

5. A method of classifying a sample as derived from an individual having schizophrenia or at risk for developing schizophrenia comprising the steps of:
a) isolating a gene expression product from at least three informative genes shown in FIG. 3 from one or more cells in said sample; and
b) determining the expression of at least three informative genes,
wherein if said informative genes are expressed at a reduced level in the sample as compared with a normal control, the sample is classified as derived from an individual having schizophrenia or at risk for developing schizophrenia.

6. A method of classifying a sample as derived from an individual having bipolar disorder or at risk for developing bipolar disorder comprising the steps of:
a) isolating a gene expression product from at least three informative genes shown in FIGS. 4 and 5 from one or more cells in said sample; and
b) determining the expression of at least three informative genes,
wherein if said informative genes are expressed at an increased level in the sample as compared with a normal control, the sample is classified as derived from an individual having bipolar disorder or at risk for developing bipolar disorder.

7. A method of identifying a compound for use in modulating a psychiatric disorder, comprising the steps of:
a) providing a cell or cell lysate sample;
b) contacting the cell or cell lysate sample with a candidate compound; and
c) detecting an increase in expression of at least one informative gene having decreased expression in an individual having a psychiatric disorder,
wherein a candidate compound that increases the expression of the informative gene is a compound for use in modulating the psychiatric disorder.

8. A method of identifying a compound for use in modulating a psychiatric disorder, comprising the steps of:
a) providing a cell or cell lysate sample;
b) contacting the cell or cell lysate sample with a candidate compound; and
c) detecting a decrease in expression of at least one informative gene having increased expression in an individual having a psychiatric disorder,
wherein a candidate compound that decreases the expression of the informative gene is a compound for use in modulating the psychiatric disorder.

9. A method for modulating a psychiatric disorder in an individual comprising down-regulating in the subject at least one informative gene shown to be expressed, or expressed in increased levels as compared with a control, in individuals having a psychiatric disorder or at risk for developing a psychiatric disorder.

10. A method for modulating a psychiatric disorder in an individual comprising up-regulating in the subject at least one informative gene shown not to be expressed, or expressed at reduced levels as compared with a control, in individuals having a psychiatric disorder or at risk for developing a psychiatric disorder.

11. A method of assessing treatment efficacy in an individual having a psychiatric disorder comprising determining the expression level of one or more informative genes at multiple time points during treatment, wherein a decrease in expression of the one or more informative genes shown to be expressed, or expressed at increased levels as compared with a control, in individuals having a psychiatric disorder or at risk for developing a psychiatric disorder, is indicative that treatment is effective.

12. A method of assessing treatment efficacy in an individual having a psychiatric disorder comprising determining the expression level of one or more informative genes at multiple time points during treatment, wherein an increase in expression of the one or more informative genes shown not to be expressed, or expressed at reduced levels as compared with a control, in individuals having a psychiatric disorder or at risk for developing a psychiatric disorder, is indicative that treatment is effective.

13. A method of classifying a sample as derived from pre-frontal cortex or cerebellum comprising the steps of:
a) isolating a gene expression product from at least three informative genes from one or more cells in said sample; and
b) determining the expression of at least three informative genes,
wherein said expression is correlated with a sample derived from pre-frontal cortex or cerebellum, thereby classifying the sample.

14. Oligonucleotide microarrays having immobilized thereon a plurality of oligonucleotide probes specific for one or more informative genes selected from the group consisting of the genes in FIGS. 1A-1D, 2A-2E, 3, 4 and 5.
